(19) United States
(12) Patent Application Publication
(10) Pub. No.: US 2026/0157616 A1
(43) Pub. Date: Jun. 11, 2026

(54) MEDICAL DEVICE PROCESSOR

(71) Applicant: American Sterilizer Company, Mentor, OH (US)

(72) Inventors: Sandeepan DUTTA, Lyndhurst, OH (US); John LADA, Chardon, OH (US); Ethan K. LANGE, Painesville, OH (US); Brian T. MILLIFF, Kirtland, OH (US); Michael RABINOVICH, Solon, OH (US)

(73) Assignee: American Sterilizer Company, Mentor, OH (US)

(21) Appl. No.: 19/150,109

(22) PCT Filed: Jan. 5, 2024
(86) PCT No.: PCT/US2024/010396

§ 371 Date: Jul. 22, 2025

Related U.S. Application Data
(60) Provisional application No. 63/485,784, filed on Feb. 17, 2023.

(51) Int. Cl. A61B 1/12

(52) U.S. Cl. CPC A61B 1/125; A61B 1/123

(57) ABSTRACT

A basin assembly for a medical device processor includes a basin tub comprising a bottom surface, one or more side surfaces, and a raised island. The one or more side surfaces define a first side, second side opposed the first side, third side connecting the first and second sides, and fourth side connecting the first and second sides. The basin tub includes one or more lumen connector ports proximate the third side, a control handle area proximate an intersection of the first and third sides, a light guide connector area located proximate an intersection second and third sides, a plug area is located on a top surface of the raised island, and a tube/cord area located proximate the second and fourth sides and surrounding at least a portion of the raised island. In some embodiments, the medical device processor includes one or both of variable speed pump(s) and a strainer.

### FIELD OF INVENTION

This application relates generally to a medical device processor and processing method, and more particularly to a medical device processor and processing method for cleaning, disinfecting, and/or sterilizing one or more medical devices.

### BACKGROUND

Medical device processors, also referred to as medical instrument processors, are widely used in various health care settings and are typically associated with high-level disinfection or sterilization of medical instruments. An example of such a processor is an automated endoscope reprocessor (AER) used for reprocessing endoscopes, such as duodenoscopes, and endoscope accessories. AERs are designed to kill microorganisms in or on reusable endoscopes by exposing their outside surfaces and interior channels to liquid chemical sterilant or high-level disinfectant solutions.

Liquid chemical AERs include a tray or basin to house the medical device as the disinfection/sterilization processing cycle is performed. The tray or basin may be shaped and sized to accept and hold a specific type of medical device. While it may be desired to process different medical devices with the same AER, one challenge that is presented is the widely variable form factor that is encountered even among a particular type of medical device. For example, considering the range of different endoscopy procedures and device manufacturers, endoscopes have a complex and widely variable form factor. The form of existing processing trays and basins have been designed to accommodate as many different endoscopes as possible, but there are shortcomings.

Smaller basins can limit endoscope placement variation, but ultimately limit the ability to accommodate large devices, such as endoscopes with large ultrasound plugs. Larger basins can accommodate a larger variety of endoscopes due to the additional volume provided by the basin, but they also allow for or require variation in endoscope placement/orientation. While this may be acceptable for reprocessors that perform high-level disinfection, it is not acceptable for reprocessors performing sterilization. This is because efficacy testing is performed at specific high-risk contact sites on the scope exterior, and these contact site locations would change with scope placement/orientation variation. Conventionally, interchangeable trays for different types of endoscopes are used and need to be changed between cycles if processing multiple endoscope types in a sterile processing department or endoscopy suite. Different prescribed placement schemes for different endoscopes are also commonly employed, requiring the user to reference instructions or a wall chart. This can be burdensome and introduce the potential for operator error.

The variation in medical devices processed in the medical device processor also presents challenges in the processing of the instrument. For example, AERs use fluid media to clean and disinfect/sterilize internal endoscope lumens/channels. The fluid is moved around and through the device using adequately sized in line pump(s) to ensure effective cleaning and disinfection/sterilization. Conventionally these pumps are fixed speed pumps that cannot adjust to differing device geometry. For example, endoscope channels/lumens can vary from 0.8 mm to 4.2 mm or larger. This leads to higher internal pressure for endoscopes with smaller lumen diameters and lower than minimum set pressures for endoscopes with larger lumen diameter. Most AER manufacturers use a custom manifold design and/or inline flow /ducers/ restrictors designed per endoscope (lumen geometry and number of lumens) to ensure that the pumps of a given AER can maintain pressure for endoscopes being processed. While various custom manifolds can be used for different endoscopes, considering there are thousands of endoscopes available in the market, it is not practical for a user to have custom manifolds for all available endoscopes.

In addition, the push to design the AER to accommodate as many different endoscopes as possible, developments in the endoscope reprocessing field have also pushed manufacturers to develop AERs that integrate endoscope cleaning in addition to disinfection/sterilization. However, very few AERs perform effective high-impingement cleaning, including removal of soil from the endoscope, in addition to high-level disinfection. Moreover, no AER has been found on the market that performs both high-impingement cleaning and sterilization.

One challenge that arises is the straining of processing fluid used during the cycles of the medical device. A strainer is needed to ensure that the channels, spray bars, and nozzles of the AER do not clog due to debris on the endoscope being processed. Some conventional AERs use a strainer with a large opening, enough to block objects like pens, pencils or earrings from getting into the system. Also, most of the products include a removable strainer. The operator should remove and clean the strainer after processing an endoscope. Based on the size of the strainer and expected debris load, the operator may need to clean the strainer after every cycle or at the end of the day. These strainers are usually in-line with the fluid flow with openings less than the smallest opening in the fluid path (e.g., spray bar or nozzle openings in the basin). The cleaning and sterilization/disinfection process leads to accumulation of contaminated debris such as tissues, coagulated blood, and fibers, on the strainer. In addition to reducing flow and providing stress on the system, the clogged debris has the potential to contaminate the device being processed, particularly because conventional strainers are, at best, cleaned after a given processing cycle is complete. Both fixed and removable strainers retain debris during the cycle, and this causes debris to float back towards the endoscope during a subsequent fluid fill phase, and may cause the debris to remain on the surface of the endoscope at the end of the cycle. Both fixed and removable strainers, if not cleaned due to operator error or some other issue, can cause the reprocessor to clog and could compromise efficacy of the sterilization/disinfection cycle. Debris accumulated on the strainer over multiple processing cycles could also lead to cross contamination between different endoscopes. Cleaning of the strainers also can be difficult or cumbersome, and leaves open the possibility of user error, such as an operator forgetting to clean the strainer or an operator forgetting to replace the strainer once removed.

Accordingly, there remains a need for further contributions in this area of technology.

### SUMMARY OF INVENTION

The application relates to a medical device processor having a basin within which the medical device is processed. The medical device processor may conduct an automated endoscope cleaning and disinfection/sterilization cycle.

In some embodiments of the medical device processor, the basin can accommodate several different types of endoscopes. The interior volume of the universal open basin inventive concept is geometrically optimized such that the endoscopes processed by the processor, regardless of size, type, and manufacturer, can be accommodated in a standardized placement and orientation. As such, the basin may be considered a “universal” basin. The basin volume is laid out with distinct placement areas for the endoscope control handle, light guide connector, ultrasound plug, and insertion tube (and other cords). Furthermore, these regions are identified with raised endoscope placement icon features which provide guidance to the user when placing an endoscope in the basin. The layout of the basin and the icon features effectively controls and guides placement in the basin.

In some embodiments of the medical device processor, the basin is also configured for both high-impingement cleaning and disinfection/sterilization. The raised features (scope offset bumps) on the basin floor and side walls enhance the efficacy of both the sterilization and cleaning processes by minimizing contact between the endoscope and basin.

In some embodiments of the medical device processor, variable speed pump(s) are controlled to provide a predetermined pressure in connection with the fluid flow into the channels/lumens of the device. The design is independent of endoscope type (diameter of endoscope lumens/channels and number of endoscope lumens/channels), and also will work for endoscopes that may be released in the future. The design may reduce the overall number of custom manifolds and/or inline flow reducers/restrictors, and may eliminate the need for requalification or recertification of the AER for purposes of processing new devices.

In some embodiments of the medical device processor, a self-cleaning strainer is included in-line with the drain of the basin. With the design of the self-cleaning strainer, its arrangement in the manifold, and the operation of the AER, the strainer is cleaned every time the unit fills with water. This eliminates dependency on the operator to clean the strainer, during and after the cycle. The arrangement of the strainer and the respective inputs/outputs in the manifold help to eliminate debris caught by the strainer in a given cycle, and helps to eliminate cross contamination between cycles.

In accordance with one aspect of the present application, a basin assembly for a medical device processor, includes: a basin tub including a bottom surface, one or more side surfaces, and a raised island extending from the bottom surface, the bottom surface, the one or more side surfaces, and the raised island defining an interior volume of the basin tub, wherein: the one or more side surfaces define a first side, a second side opposed the first side, a third side connecting the first and second sides, and fourth side connecting the first and second sides and opposed the third side; and the basin tub includes: one or more lumen connector ports proximate the third side; a control handle area proximate an intersection of the first and third sides, the control handle area shaped so that a control handle disposed in the control handle area is oriented with its ports proximate the lumen connector ports; a light guide connector area located proximate an intersection second and third sides, the light guide connector area shaped so that a light guide connector disposed in the light guide connector area is oriented with its light guide prong distal the internal lumen connector ports; a plug area is located on a top surface of the raised island; and a tube/cord area located proximate the second and fourth sides and surrounding at least a portion of the raised island.

In some embodiments, the basin assembly includes a drain extending through the bottom surface of the basin tub located in the control handle area.

In some embodiments, the basin assembly includes raised portions at the bottom surface of the basin tub in at least one of the control handle area, light guide connector area, or tube/cord areas.

In some embodiments, the basin assembly includes raised portions at the one or more side surfaces of the basin tub.

In some embodiments, a height of the raised features is within a range of 3 mm to 15 mm, a width of the raised features is within a range of 5 mm to 25 mm, and a radius of the raised features is within a range of 3 mm to 15 mm.

In some embodiments, a top surface of the raised island is a contoured surface configured to retain a plug.

In some embodiments, the basin assembly includes: a control handle icon located at the bottom surface in the control handle area indicating a control handle and an orientation at which a control handle is to be disposed in the control handle area; a light guide connector icon located at the bottom surface in the light guide connector area indicating a light guide connector and an orientation at which a light guide connector is to be disposed in the control handle area; and a plug icon located at the raised island surface in the plug area indicating a plug and an orientation at which a plug is to be disposed in the plug area. In some embodiments, at least one of the control handle icon, light guide connector icon, or plug icon are raised icons formed as a part of the basin tub surface. In some embodiments, at least one of the control handle icon, light guide connector icon, or plug icon are recessed icons formed as a part of the basin tub surface. In some embodiments, at least one of the control handle icon, light guide connector icon, or plug icon are printed, painted, or adhered to the basin tub surface.

In some embodiments, the basin assembly includes a vent port extending through the side wall of the basin tub, the vent port including: a vent wall including first and second opposed surfaces and holes extending therethrough; an elastomeric umbrella-type diaphragm valve mounted to the vent wall and in contact with the first opposed surface distal the interior volume of the basin tub; a shielding extending from the second opposed surface, the shielding including a shielding wall and a side wall extending from the shielding wall and connecting the shielding wall to the vent wall, wherein the shielding wall is spaced apart from the vent wall and covers the holes when the basin vent port is viewed in a direction orthogonal to the second opposed surface of the vent wall.

In some embodiments, the basin assembly includes a vacuum break port extending through the side wall of the basin tub, the vacuum break port including: a vent wall including first and second opposed surfaces and holes extending therethrough, the first opposed surface distal the interior volume of the basin tub, the second opposed surface proximate the interior volume of the basin tub; an elastomeric umbrella-type diaphragm valve mounted to the vent wall and in contact with the second opposed surface; and a shielding extending from the second opposed surface, the shielding including a shielding wall and a side wall extending from the shielding wall and connecting the shielding wall to the vent wall, wherein the shielding wall is spaced apart from the vent wall and covers at least a portion of the diaphragm as viewed in a direction orthogonal to the second opposed surface of the vent wall, wherein the shielding wall includes a cutout that exposes a portion of the diaphragm when viewed in the direction orthogonal to the second opposed surface of the vent wall.

In accordance with another aspect of the present disclosure, a method of processing a medical device includes: performing a cycle in a medical device processor, the medical device processor including: a basin assembly including a basin tub and one or more connectors configured to connect to a medical device processed in the medical device processor; a supply line connected to the one or more connectors for supplying fluid to the medical device; a variable speed pump connected to the supply for pumping the fluid; a pressure sensor downstream from the variable speed pump and configured to detect a pressure of the fluid pumped from the variable speed pump to the one or more connectors; and a controller coupled to the variable speed pump and the pressure sensor and configured to control the speed of the variable speed pump; controlling the variable speed pump to operate at a first predetermined speed; determining that a pressure detected by the pressure sensor is above or below a predetermined value or outside of a predetermined range during the cycle; and controlling the variable speed pump to operate at a second predetermined speed.

In some embodiments, the medical device processor includes a lumen manifold including the one or more connectors.

In accordance with another aspect of the present disclosure, a method of processing a medical device includes: starting a cycle in a medical device processor, the medical device processor including: a basin assembly including a basin tub, one or more fluid inputs, and a basin drain; a manifold including a front manifold portion and a back manifold portion coupled to the front manifold portion, the front manifold portion and the back manifold portion forming a flow path therethrough, and a strainer disposed between the front manifold portion and the back manifold portion in the flowpath; a return line connecting the basin drain to the front manifold portion; and a supply line connecting the back manifold portion to the basin assembly fluid inputs; wherein the front manifold portion includes a drain port and the rear manifold portion includes a water input port; introducing water into the water input port; maintaining the drain port in an open position for a predetermined amount of time so that water input from the water input port flows through the strainer in a first direction toward the drain port and exits the manifold; closing the drain port after the predetermined amount of time; closing the water input port after a predetermined amount of water is introduced; and conducting the cycle wherein the flow of water through the manifold is in a second direction opposite the first direction.

In some embodiments, the method includes draining the water via the drain port upon completion of the cycle.

In some embodiments, the one or more fluid inputs of the basin includes one or more nozzles, and one or more connectors configured to connect to a medical device processed in the medical device processor.

In some embodiments, the front manifold portion incudes one or more inlet ports for inputting one or more of detergent solution, disinfectant, or sterilization solution.

In some embodiments, the back manifold portion includes one or more outlet ports connectable to the one or more fluid inputs of the basin assembly.

In some embodiments, the strainer is a metal mesh strainer.

In some embodiments, the strainer is removably housed between the front manifold portion and the back manifold portion.

The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention.

FIG. 1 is a schematic perspective view of an exemplary medical device processor.

FIG. 2A is a schematic view of parts of an exemplary medical device processor.

FIG. 2B is a schematic view of parts of an exemplary medical device processor.

FIG. 3 is a schematic perspective view of parts of an exemplary basin assembly.

FIGS. 4-6 are schematic top views of parts of an exemplary basin assembly.

FIG. 7 is a schematic cross-sectional view of an exemplary raised feature.

FIG. 8 is a schematic perspective view of an exemplary manifold

FIG. 9 is a schematic perspective view showing a cross-section of the exemplary manifold of FIG. 8.

FIG. 10 is a schematic top view of parts of an exemplary basin assembly.

FIG. 11 is a schematic front perspective view of an exemplary basin vent port.

FIG. 12 is a schematic rear perspective view of an exemplary basin vent port of FIG. 10.

FIG. 13 is a schematic front perspective view of an exemplary vacuum break port.

FIG. 14 is a schematic rear perspective view of an exemplary vacuum break port of FIG. 13.

FIG. 15A is a schematic perspective view of an exemplary manifold.

FIG. 15B is a schematic side view of an exemplary manifold.

FIG. 16 is a schematic view of an exemplary control system.

FIG. 17 is a flowchart showing an exemplary process including both cleaning and disinfecting/sterilizing cycles.

FIG. 18 is a flowchart showing an exemplary process of adjusting pressure.

FIG. 19 is a schematic view of parts of an exemplary medical device processor.

FIG. 20 is a flowchart showing an exemplary straining process.

FIGS. 21A-21C are schematic views of exemplary flows of fluid in the manifold in accordance with the process of FIG. 20.

### DETAILED DESCRIPTION

While the present invention can take many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications of the described embodiments, and any further applications of the principles of the invention as described herein, are contemplated as would normally occur to one skilled in the art to which the invention relates.

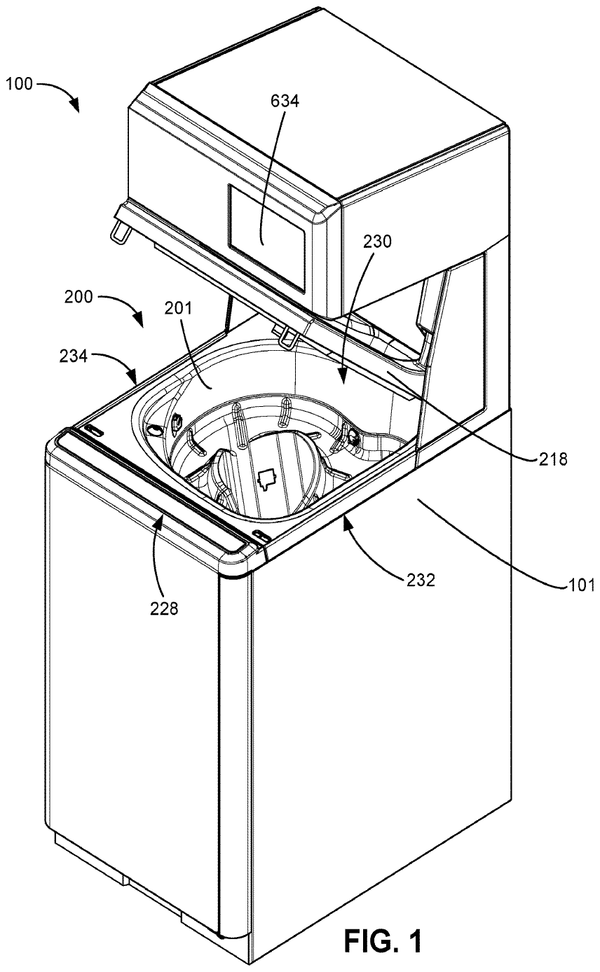

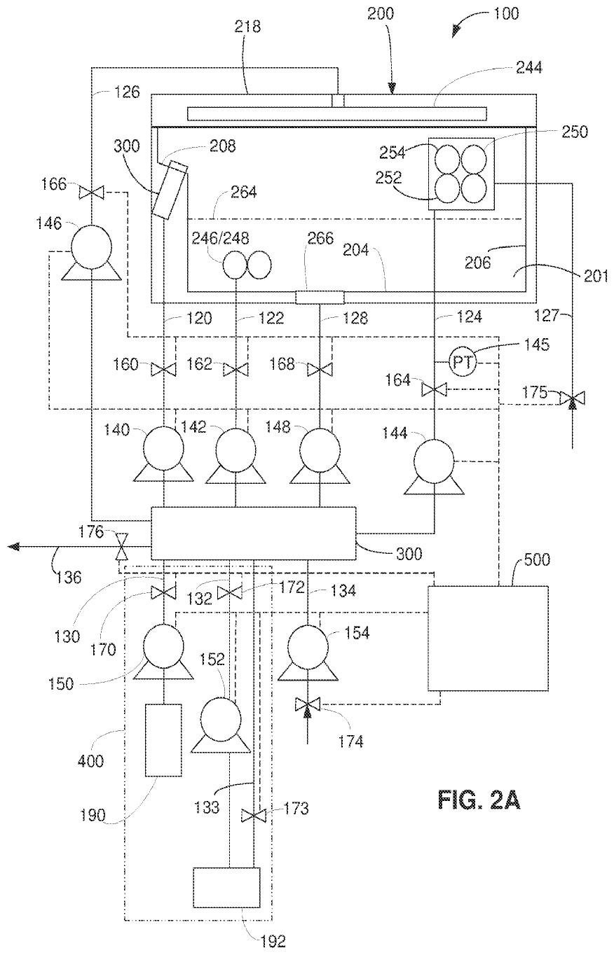

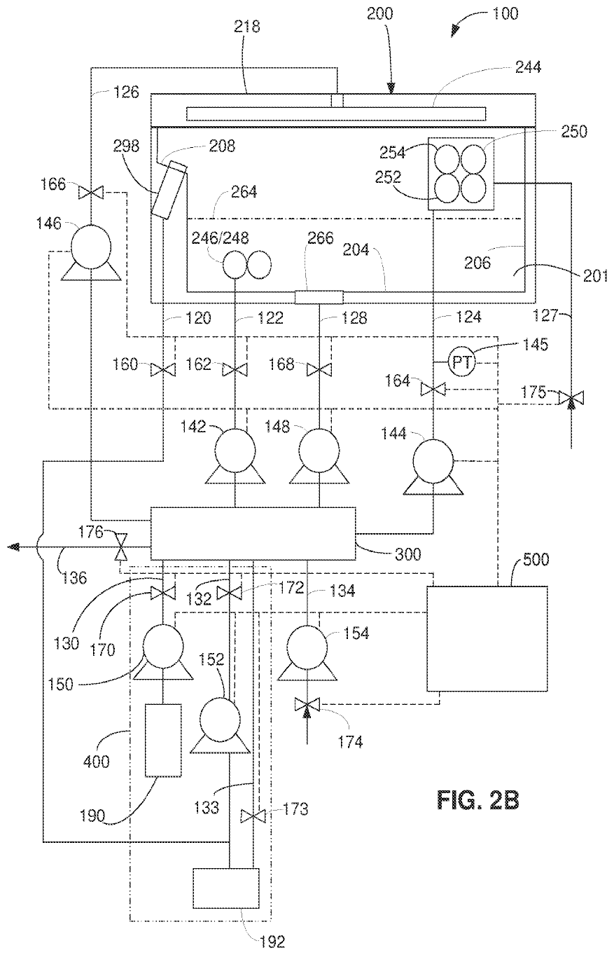

Turning now to the drawings, and initially to FIGS. 1 and 2A, an exemplary medical device processor is shown at 100. The medical device processor 100 is configured to clean and decontaminate one or more medical devices inserted into the medical device processor 100. The medical device processor 100 may be any type of system for cleaning and decontaminating medical devices or instruments, for example, by a cleaning process and a high-level disinfection process and/or sterilization process. A high-level disinfection process and/or sterilization process will also be generally referred to herein as a decontamination process. In an exemplary embodiment, the medical device processor 100 is an automated endoscope reprocessor (AER) used for reprocessing endoscopes, such as duodenoscopes, and endoscope accessories.

In the exemplary embodiment shown, the medical device processor 100 includes a basin assembly 200, manifold 300, chemical dosing system 400, and a control system 500. As shown in FIG. 1, the medical device processor 100 includes a housing 101 that retains components of the system. In the embodiment shown, the processor is embodied as a single basin system. In other embodiments, it will be appreciated that the medical device processor may include more than one basin, and/or the medical device processor may be embodied as a passthrough configuration (two-sided) medical device processor.

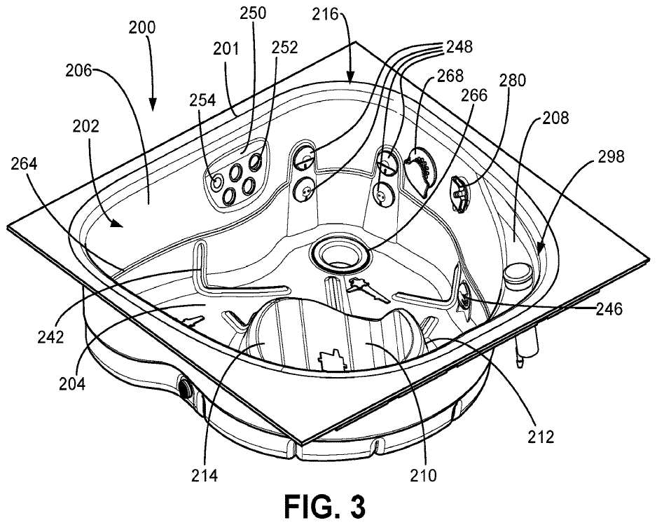

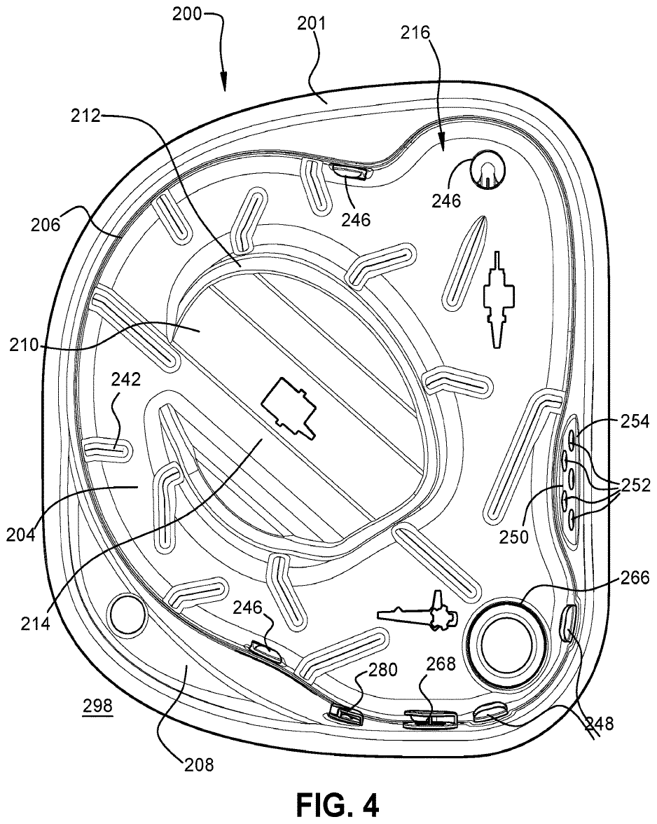

The basin assembly 200 is configured to house the one or more medical devices for purposes of performing the cleaning and high-level disinfecting and/or sterilizing. As shown, and with additional reference to FIGS. 3 and 4, the basin assembly 200 includes a basin tub 201 having an opening 202 (open top) for receiving a medical device for processing. The basin tub 201 includes a bottom surface 204 and one or more side walls 206. The one or more side walls 206 extend from the bottom surface 204 and define a height/depth of the basin tub 201. In the embodiment shown, the basin includes a step surface 208. The step surface 208 is connected to the side wall 206. In the example shown, the basin tub 201 also includes a raised island 210 extending from the bottom surface 204. The raised island 210 includes one or more side surfaces 212 and a top surface 214.

The basin tub 201 defines an interior volume 216 within which the one or more medical devices and liquid solution including detergent, high-level disinfectant solution, or chemical sterilant may be disposed during processing of the one or more medical devices. More specifically, the surfaces of the basin tub 201, including the bottom surface 204, one or more side walls 206, step surface 208, and raised island 210 collectively define the interior volume 216. A lid 218 (FIGS. 1 and 2A) or door may enclose the interior volume 216 of the basin tub 201 from the environment.

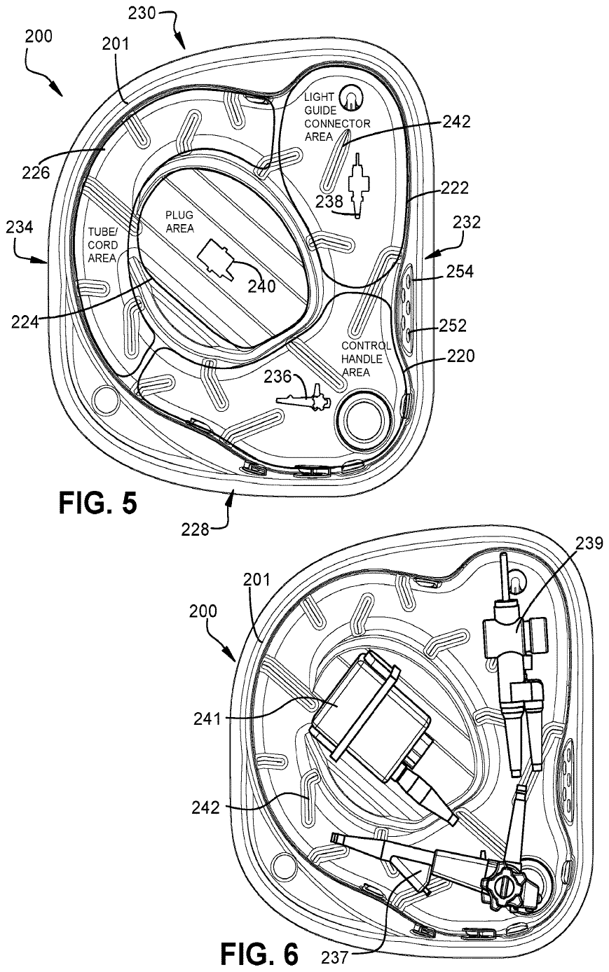

With additional reference to FIG. 5, the basin tub 201 includes four main areas: A control handle area 220, a light guide connector area 222, a plug area 224, and a tube/cord area 226. As shown in FIG. 6, these areas 220, 222, 224, 226 correspond to the main components of an endoscope. The areas can accommodate most endoscopes, regardless of size, type, and manufacturer, in a standardized placement and orientation.

As shown in FIG. 1, the basin 201 may be arranged in the medical device processor 100 such that a user operating the medical device processor is closest to the side of the basin assembly proximate the control handle area 220 and the step surface 208. As such, this side of the basin may be referred to as the front side 228 of the basin. The other sides of the basin assembly may be referred to as the rear side 230, right side 232, and left side 234. Because the orientation of the basin may vary, the sides may also be referred to as the first side 228, second side 230, third side 232, and fourth side 234, respectively.

With reference to FIG. 5, the control handle area 220 is located proximate the front-right corner of the basin tub 201 and is proximate the lumen connector ports 252. The control handle area 220 is shaped so that a control handle disposed in the control handle area is oriented with its ports proximate the lumen connector ports 252. As such, the control handle can be ergonomically positioned so the user has easy access to make the requisite connections between the lumen(s) of the control handle and the lumen connector ports 252. The light guide connector area 222 is located proximate the rear-right corner of the basin tub 201. The light guide connector area 222 is shaped so that a light guide connector disposed in the light guide connector area is oriented with its light guide prong distal the internal lumen connector ports 252. For ergonomic purposes, because the light guide connector only rarely requires internal lumen flow unit connections, this area is located further from the connectors. The plug area 224 is located on the top surface 214 of the island 210, elevated from the bottom surface 204. As shown, for example in FIG. 3, the top surface 214 of the island 210 is contoured so as to accept and retain a plug. The tube/cord area 226 surrounds the left side and rear side of the island 210 and plug area 224.

It will be appreciated that in other embodiments, the orientation of the areas 220, 222, 224, 226 of the basin tub 201 can be adjusted relative to the front side 228, rear side 230, right side 232, left side 234. For example, the orientation can be rotated a specified degree relative to the orientation shown in the figures (e.g., 90°, 180°, 270°). In addition or alternatively, the orientation of the basin tub 201 can be a mirror image of that shown in the figures.

The component placement regions are identified in the basin tub 201 using icon features. As shown, a control handle icon 236 is located in the control handle area 220; a light guide connector icon 238 is located in the light guide connector area 222; and a plug icon 240 is located in the plug placement area 224. This provides guidance to the user on endoscope placement/orientation without requiring any supplemental reference materials such as instructions or wall charts. The icon features 236, 238, 240 are respectively oriented to show the orientation that the components of the endoscope are to be placed in the basin 201. As exemplified in FIG. 6, a representative endoscope control handle 237, light guide connector 239, and plug 241 are oriented in the basin tub 201, similar to the orientation shown by the icons. For purposes of clarity, the insertion tube and other cords are not depicted.

In the exemplary embodiments shown in the figures, the icon features are raised icons relative to the surface of the basin. In other embodiments, the icon features are recessed relative to the surface of the basin. In those embodiments in which the basin is formed by a molding process, the raised or lowered icons may be a part of the mold and formed as part of the molding process. In other embodiments, the icon features are printed, painted, or otherwise adhered to the basin surface.

With continued reference to FIGS. 3-6, the basin tub 201 includes raised features 242, which may also be referred to as scope offset bumps. In the example shown, the raised features 242 are located at the bottom floor and/or the walls of the basin. The raised features 242 assist in minimizing contact area between the endoscope and the basin surfaces (e.g., bottom surface 204 and side walls 206). Minimizing contact area between the endoscope and basin surfaces allows for soil removed from the scope to more freely reach the drain. During the cleaning phase of the processing cycle, soil is removed from the scope. If the endoscope is making excessive contact with the basin, soil can become lodged between the endoscope and basin surface, and not drain out of the basin tub 201 properly. By offsetting the endoscope from the bottom surface 204 and side walls 206, the soil can freely drain out of the basin tub 201. The raised features 242 also can improve sterilization efficacy on the exterior surfaces of the medical device. During the sterilization phase of the processing cycle, sterilant is circulated in the basin around the surface of the endoscope. If the endoscope is making excessive contact with the basin, small air bubbles could remain static on an exterior surface of the endoscope, inhibiting sterilization efficacy at that surface site. By offsetting the endoscope from the bottom floor and side walls, the sterilant can flow more uniformly around the endoscope, thereby improving sterilization efficacy on the exterior surfaces of the endoscope. In the exemplary embodiment shown, the raised features 242 are located in each of the control handle area 220, the light guide connector area 222, and the tube/cord area 226. The top surface 214 of the island 210 in the plug area 224 is contoured and may allow for drainage and improved sterilization efficacy of the plug placed thereon. In other embodiments, raised features 242 can also be located in the plug area.

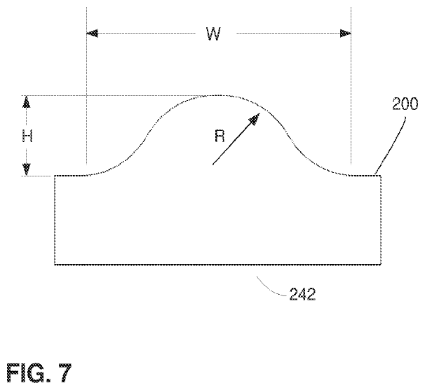

A cross-sectional view of a raised feature as viewed along the length of the raised portion is shown in FIG. 7. The raised features have a height H, a width W, and a radius R, and extend along a length (normal to the plane of the page). As shown in the figures, some of the raised features extend linearly and are linear shapes along their length. In other embodiments, at least some of the raised features include linear portions and curved portions. In still other embodiments, at least some of the raised features may curve along their entire length. The height H, width W, and radius R of the raised features 242 may be provided to provide sufficient clearance of the endoscope from the surfaces of the basin while still allowing for sufficient volume within the basin and sufficient flow of the fluid within the basin toward the drain. In some examples, the height H of the raised features 242 is within a range of 1 mm to 20 mm. In other examples, the height H of the raised features is within a range of 3 mm to 15 mm. In other examples the height H of the raised features is 5 to 10 mm. In some examples, the width W of the raised features is within a range of 5 mm to 25 mm. In other examples, the width W of the raised features is within a range of 10 mm to 20 mm. In other examples the width W of the raised features is 10 to 15 mm. In some examples, the radius R of the raised features is within a range of 1 mm to 20 mm. In other examples, the radius R of the raised features is within a range of 3 mm to 15 mm. In other examples the radius R of the raised features is 5 to 10 mm. In one example, the height H of the raised features is about 5.7 mm, the width W of the raised features is about 21.3, and the radius R of the raised features is about 6 mm.

With continued reference to FIGS. 2 and 3, the basin assembly 200 includes one or more spray bars 244, one or more circulation nozzles 246, and/or one or more control handle nozzles 248 for spraying the endoscope with the liquid solution including detergent, high-level disinfectant solution and/or chemical sterilant during the cleaning process, high-level disinfecting process, and/or sterilizing process. In some embodiments, the spray bar 244 is a rotatable spray bar that rotates during the cleaning process, high-level disinfecting process, and/or sterilizing process.

The circulation nozzles 246 act as basin inlets to circulate fluid into the basin tub 201 and around the endoscope, assisting with cleaning and sterilization. In the exemplary embodiment shown, the basin includes three circulation nozzles 246. Two of the nozzles 246 are located on the sidewall of the basin, one at the front and one at the rear. One of the circulation nozzles 246 is located on the basin floor 204 in the rear-right corner. It will be appreciated that in other embodiments, the basin may include more or fewer circulation nozzles 246. It will also be appreciated that the circulation nozzles may be located in different respective positions than what is shown in the exemplary embodiment.

The control handle nozzles 248 are located on the sidewall 206 of the basin tub 201 at the control handle area 220. The control handle nozzles are configured as spray nozzles and may assist the spray bar with cleaning, disinfecting, and/or sterilizing the outer surface of the control handle.

It will be appreciated that in some embodiments, the one or more spray bars 244, one or more circulation nozzles 246, and/or one or more control handle nozzles 248 may also be used to introduce gas (e.g., compressed air) into the basin, for example, for assisting in keeping the basin at positive pressure while draining the liquid from the basin.

The basin assembly 200 includes lumen connector ports 252 and leak test port 254. The lumen connector ports 252 serve to connect interior channel(s) of a medical device to a source of detergent, high-level disinfectant solution, or chemical sterilant. The lumen connector ports 252 may also serve to connect the interior channel(s) of a medical device to a gas source, for example, in embodiments where compressed gas (e.g., air) is used to clear and/or dry the interior channel(s) as part of the processing. The leak test port 254 serves to connect the endoscope to an air source. In the example shown, the lumen connector ports 252 are located on the right side wall of the basin, slightly biased toward the front of the basin. As described above, this location provides for optimized attachment of the interior channels of the control handle as oriented in the intended position. In an example in which the medical device is an endoscope, the endoscope may be placed in the basin tub 201 and interior channels of the endoscope may be respectively connected to lumen connector ports 252 and leak test port 254. A leak test may be performed by passing air through the leak test port 254 into the endoscope. During the cleaning process, high-level disinfecting process, and/or sterilizing process, the interior channels of the endoscope may be exposed to detergent, high-level disinfectant solution, and/or liquid chemical sterilant to clean surfaces of the endoscope and to kill microorganisms in or on the endoscope.

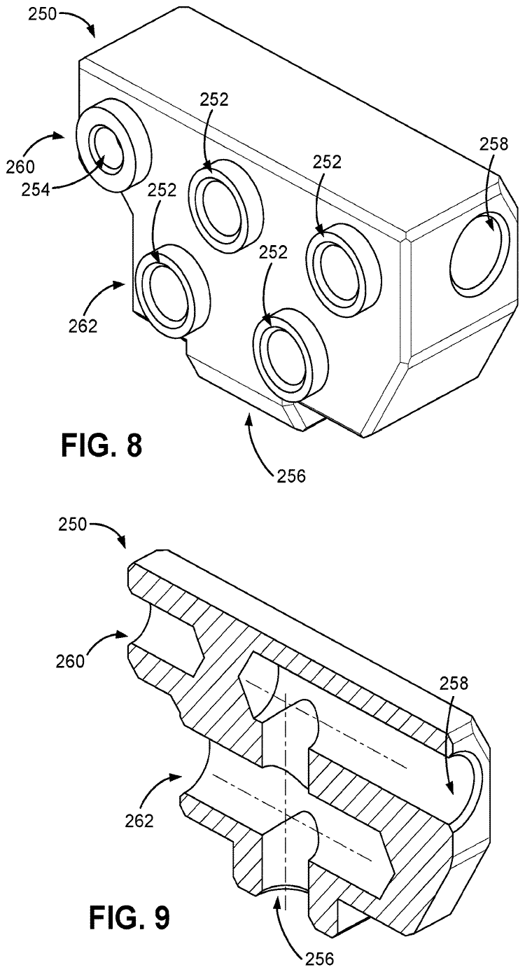

With additional reference to FIGS. 8 and 9, the lumen connector ports 252 and leak test port 254 are included as part of a lumen manifold 250 included as part of the basin assembly 200. The lumen manifold 250 includes ports to support endoscopes with one or multiple internal lumens/channels. As shown, the lumen manifold 250 includes lumen connector ports 252, as well as a leak test port 254.

The connector manifold also includes leak test gas input 260 and input ports 256, 258, 262. In some embodiments, one or more of the input ports 256, 258, 262 may be an input port for inputting liquid to the manifold. In some embodiments, one or more of the input ports 256, 258, 262 may be an input port for inputting gas to the manifold. In some embodiments, one or more of the input ports 256, 258, 262 may be a port for monitoring pressure of the fluid, and in such embodiments may be alternatively referred to as a pressure monitoring port. It will be appreciated that in some embodiments, the different ports 256, 258, 262 may have different respective functionalities. For example, in one embodiment, input port 256 is a liquid input, input port 258 is a gas input and input port 262 is a pressure monitoring port. It will further be appreciated that in some embodiments, one or more of the input ports 256, 258, 262 may be plugged. For example, in one embodiment, input ports 258 and 262 are plugged and input port 256 is a liquid input port. The specific configuration of the input ports 256, 258, 262 can vary depending on the specific implementation of the manifold in the system. As shown by the internal cross-section of the lumen manifold in FIG. 9, the lumen connector ports 252 are internally connected to one another, such that they are provided similar pressure. The input ports 256, 258, 262 are also in fluid communication with the lumen connector ports 252.

With continued reference to FIGS. 2 and 3, the basin assembly 200 includes a fill line 264. The fill line 264 designates the height at which the solution may be filled in the basin tub 201 during processing. The basin tub 201 is configured such that not all internal surfaces are immersed. Parts of the side wall(s) of the basin tub 201 and/or other surfaces are above the fill line and not submerged when the basin is filled as part of the processing. Directed spray from the spray bar and/or nozzles is used to disinfect/sterilize non-immersed surfaces of the medical device being processed, as well as the non-immersed surfaces of the basin.

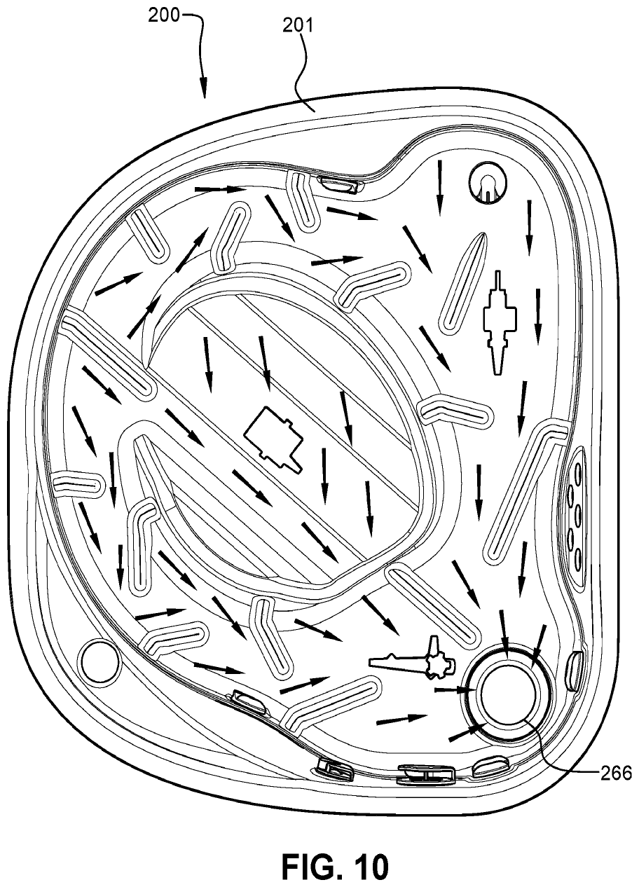

The basin assembly 200 includes a drain 266 for draining the solution including detergent, high-level disinfectant, or chemical sterilant from the basin tub 201. In the example shown, the basin is located in the front-right corner of the basin, and acts as the basin outlet. With additional reference to FIG. 10, the basin floor gradient is contoured such that the drain is located at the lowest point, to allow fluid gravity drainage as shown by the arrows. The location of the drain in the front-right corner allows for the fluid immersion level to be greatest in the control handle area 220, enhancing sterilization process efficacy. Control handles have knobs on top and are typically relatively tall compared the rest of the scope. Locating the control handle area in the deepest area of the basin can maximize submersion of the control handle. The basin outlet to the piping system can also be located away from the overhead rotating spray bar, limiting introduction of air into the medical device processor circulation pumps. The liquid exiting the basin tub 201 via the drain 266 may in some embodiments be recirculated to the basin assembly 200 (e.g., via manifold 300) during a given processing cycle. The liquid exiting the basin tub 201 via the drain 266 may also be discarded during or upon completion of a given cycle.

The basin assembly 200 includes a basin vent port 268. Because the internal basin volume is a sealed system during the processing cycle, and compressed gas may be used in some embodiments to maintain the basin at a positive pressure as it is drained, the basin vent port 268 serves as a check valve to allow gas egress and prevent over pressurization of the basin interior. In the exemplary embodiment shown, the basin vent port 268 is located on the front side wall, above the basin fill line 264.

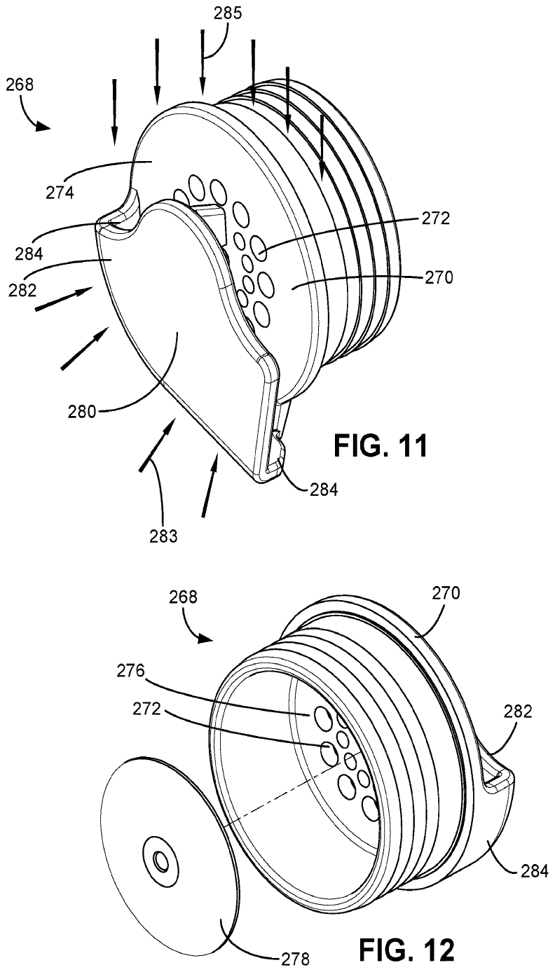

FIGS. 11 and 12 show an exemplary basin vent port 268. As shown, the basin vent port 268 includes a vent wall 270 having multiple holes 272 extending therethrough that allow the compressed air to escape the interior volume 216 of the basin tub 201. The vent wall 270 includes opposed surfaces 274, 276. As mounted to the wall of the basin tub 201, surface 274 faces the interior volume 216 of the basin tub 201 and surface 276 faces exterior to the interior volume 216. An elastomeric umbrella-type diaphragm valve 278 is mounted to and in contact with the surface 276, distal the interior volume 216 of the basin tub 201. The surface 276 serves as a seating for the diaphragm valve 278. A shielding 280 extends from the surface 274. The shielding 280 includes a shielding wall 282 and a side wall 284 that extends from the shielding wall 282 and connects the shielding wall 282 to the vent wall 270. The shielding wall 282 is spaced apart from the vent wall 270 and covers the holes 272 when the basin vent port 268 is viewed in a direction orthogonal to the surface 274 of the vent wall 270. The shielding wall 282 shields the multiple circular holes from direct spray (schematically shown by arrows 283 at the bottom of the figure), thereby limiting fluid volume loss in the basin. However, runoff from the basin side wall (schematically shown by arrows 285 at the top of the figure) can flow over the holes and shielded surfaces, then out and around the side wall. Because side wall runoff is less forceful than the direct spray, the holes 272 and shielded surfaces can be contacted by sterilant without significant loss of fluid volume in the basin. The side wall 284 is also arranged such that the side wall runoff is not collected by the basin vent port. Liquid that contacts the side wall will flow via gravity off of the side wall and to the bottom of the basin tub 201.

The basin assembly 200 includes a vacuum break port 280. During drain phases, the medical device processor may in some embodiments utilize HEPA-filtered, compressed air to maintain the basin interior at a positive pressure. However, in the case of a power failure or cycle abort, the medical device processor may be unable to introduce compressed air into the basin. Consequently, the vacuum break port 280 serves as a check valve to allow air ingress, preventing high vacuum levels in the basin and thereby allowing fluid in the basin to gravity drain. In the embodiment shown, the vacuum break port 280 is located on the front side wall, above the basin fill line.

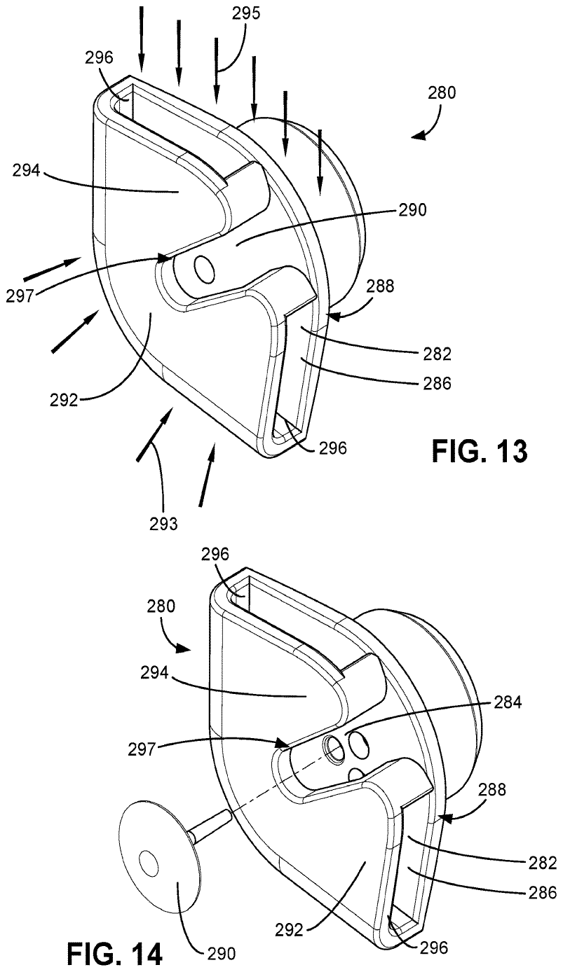

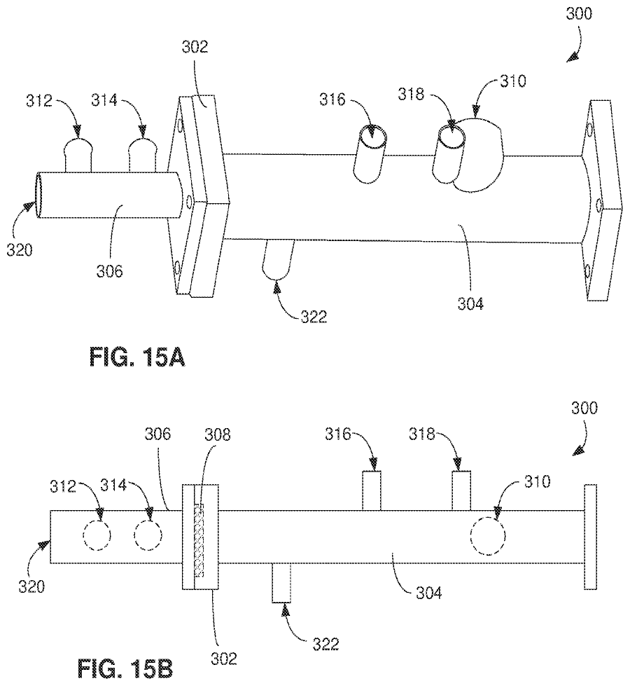

FIGS. 13 and 14 show an exemplary vacuum break port 280. As shown, the vacuum break port 280 includes a vent wall 282 having holes 284 extending therethrough that allow the air to enter the interior volume 216 of the basin tub 201. When the pressure in the basin drops below a predetermined level of vacuum, the diaphragm cracks open to allow ingress of air from the outside environment.

The vent wall 282 includes opposed surfaces 286, 288. As mounted to the wall of the basin, surface 286 faces the interior volume 216 of the basin tub 201 and surface 288 faces exterior to the interior volume. An elastomeric umbrella-type diaphragm valve 290 is mounted to the surface 286. The surface serves as a seating for the diaphragm valve 290. A shielding 292 extends from the surface 286. The shielding includes a shielding wall 294 and a side wall 296 that extends from the shielding wall 294 and connects the shielding wall 294 to the vent wall 282. The shielding wall 294 is spaced apart from the vent wall 282. The shielding wall 292 shields at least a portion of the diaphragm from direct spray (schematically shown by arrows 293 at the bottom of the figure). The shielding wall also includes a cutout 297 that allows for removal and replacement of the diaphragm 290, for example, by a qualified technician. The side wall 296 is arranged such that the side wall runoff of liquid (schematically shown by arrows 295 at the top of the figure) is not collected by the vacuum break port 280. Liquid that contacts the side wall will flow via gravity off of the side wall and to the bottom of the basin.

With continued reference to FIGS. 1-5, the basin assembly 200 includes a chamber 298 for chemical indicator (CI) strips, biological indicators (BI), and/or spore test strips. In the example shown, the chamber 298 is located in the front-left comer of the basin tub 201 at the stepped surface 208. The chamber 298 is in fluid communication with the interior volume 216 of the basin and is also connected to the manifold 300. Chemical indicator test strips, spore test strips, and/or biological indicators disposed in the chamber during a cycle are brought into contact with the high-level disinfectant solution source and/or liquid chemical sterilant to verify disinfection or sterilization efficacy. In embodiments where the medical device processor is a passthrough (two-sided) embodiment, the chamber 298 also allows for users to access the process indicators on both sides of a passthrough configuration (two-sided) medical device processor: A user can load new process indicators when starting a cycle on the “dirty” side, then another user can unload and inspect the process indicators on the “clean” side when the cycle is complete.

With continued reference to FIG. 2A, in the exemplary embodiment shown, the manifold 300 is connected to the basin assembly 200 via one or more supply lines 122, 124, 126 and one or more return lines 128. The manifold 300 is also connected to the chamber 298 via supply line 120. Pumps 140, 142, 144, 146, 148 and valves 160, 162, 164, 166, 168 are also located along the respective supply and return lines for purposes of directing fluid flow in accordance with a desired cycle. For example, supply line 126 connects the manifold 300 to the spray bar(s) 244. Pump 146 and/or valve 166 are used in the control of flow of the liquid to the spray bar(s) 244. Supply line 122 connects the manifold 300 to the nozzle(s) 246, 248. Pump 142 and/or valve 162 are used in the control of flow of the liquid to the nozzle(s) 246, 248. Supply line 124 connects the manifold 300 to the lumen manifold 250. Pump 144 and/or valve 164 are used in the control of flow of the liquid to the lumen manifold 250. Supply line 120 connects the manifold 300 to the process indicator chamber 298. Pump 140 and/or valve 160 are used in the control of flow of the liquid to the chamber 298. Supply line 128 connects the basin drain 266 to the manifold 300. Pump 148 and/or valve 168 are used in the control of flow of the liquid from the drain 266 to the manifold 300.

In some embodiments, the pump 144 is a variable speed pump. A pressure sensor 145 (e.g., pressure transducer) may be connected to supply line 124 downstream from the pump 144. As described below, the control system 500 may control the pump speed based on the pressure detected by the sensor 145. It will also be appreciated that, in some embodiments, multiple variable speed pumps or a combination of fixed speed and variable speed pumps can be used instead of a single variable speed pump 144 to control pressure of fluid to the manifold. It will also be appreciated that any other of the pumps in the system can be embodied as a fixed speed pump, variable speed pump, multiple variable speed pumps, or a combination of fixed speed and variable speed pumps.

The manifold 300 is also connected to a water supply line 134, the chemical dosing system 400, and a drain. Supply line 134 connects the water source to the manifold 300. Pump 154 and/or valve 174 are used in the control of flow of supply water to the manifold 300. The chemical dosing system includes a detergent source 190. A supply line 130 connects the detergent source 190 to the manifold 300. Pump 150 and/or valve 170 are used in the control of flow of the detergent from the detergent source 190 to the manifold 300. In some embodiments, valve 170 is a check valve. The chemical dosing system 400 includes a high-level disinfectant solution source and/or liquid chemical sterilant source 192. The high-level disinfectant and/or liquid chemical sterilant will also be referred to herein as decontaminant. In some embodiments, the decontaminant is peracetic acid and/or glutaraldehyde. Supply lines 132 and 133 form a supply path between the high-level disinfectant solution source and/or liquid chemical sterilant source 192 and the manifold 400. The supply line 132 connects the detergent manifold 400 to the high-level disinfectant solution source and/or liquid chemical sterilant source 192, and pump 152 and/or valve 172 are used in the control of flow of fluid from the manifold 400 to the high-level disinfectant solution source and/or liquid chemical sterilant source 192. Fluid can flow from the high-level disinfectant solution source and/or liquid chemical sterilant source 192 to the manifold via supply line 133. Valve 173 may be used in the control of flow of fluid from the source 192 to the manifold 400. Although not specifically shown, in other embodiments, the system may instead include a supply line and pump and/or valve arrangement similar to that shown in connection with the detergent source 190 that connects the high-level disinfectant solution source and/or liquid chemical sterilant source 192 to the manifold.

FIG. 2A also shows a supply line 127 that connects a gas source (e.g., air) to the lumen manifold 250. Valve 175 is used in the control of flow of supply of gas to the lumen manifold. The gas may be used in connection with, for example, leak testing. Although not specifically shown, the gas source may also be connected to the manifold for supplying gas to the basin via one or more of the spray bar 244, nozzles, 246, 248, and manifold 250. The gas may also be used for keeping the basin at a positive pressure during drain phases. Although not specifically shown, in some embodiment, the gas supply may also be connected to manifold 300 via a supply line. Gas supply may be used with the manifold, for example, in draining and/or drying the system and/or the lumens of the medical device.

While FIG. 2A shows a generic representation of the manifold 300, FIGS.

15A and 15B show a more detailed exemplary configuration thereof. As shown, the manifold 300 housing 302 is a two-part assembly, including a front manifold portion 304 and a back manifold portion 306. A strainer 308 is sandwiched in between the front manifold 304 and the back manifold 306. The manifold acts as a housing for the strainer. The strainer 308 is in-line with the flow path to the basin (spray bar, nozzles, lumen manifold). As shown, the front manifold portion 304 includes an input port 310 that receives fluid from the basin drain 266, and this fluid may contain debris from the endoscope. The fluid flows through the strainer 308 and the strainer retains the debris and thereby filters the water. The back manifold portion 306 includes an output port 312 connectable to the spray bar pump 244, nozzles 246, 248, and/or chamber 298, as well as an output port 314 connectable to the lumen manifold 250. The strained/filtered fluid flows from the back manifold portion 306 back through the spray bar 244, nozzles 246, 248, chamber 298, and/or internal endoscope channels (lumens). Although not specifically, shown, it will be understood that in other embodiments, the back manifold portion 306 can include additional output ports. For example, the back manifold portion 306 may include separate output ports 312 that are respectively connectable to each of spray bar 244, nozzles 246, 248, and chamber 298. In the exemplary embodiment shown, the front manifold portion 306 also includes inlet ports 316, 318 for inputting detergent solution, disinfectant and/or sterilization solution. The front manifold portion 304 also includes fluid drain port 322. The drain port 322 is arranged such that, during normal flow during a cycle, the drain port 322 is located upstream the strainer.

The water source is connected to the back manifold portion 306 via the water inlet 320. During at least a portion of a fill phase, the water inlet to the system flows through the manifold in a direction that is reverse to the normal flow direction (i.e., from the back manifold portion 304 to the front manifold portion 306). The incoming water to the unit is usually at a high pressure (e.g., 20-80 PSI). The high pressure and high flow of incoming water is used to clean the strainer. As described below, the incoming water can flow in a reverse direction through the strainer 308 and clean the strainer. The debris removed from the strainer can exit the manifold via the drain port 322. The manifold and strainer arrangement can ensure that fluid in the reprocessor is filtered during each phase, thereby helping to ensure spray bar, nozzles (in the basin or fluid flowpath), lumens or other internal channels of the endoscope are not clogged. Use of inlet water for cleaning of the strainer also helps with cleaning efficiency. The strainer can also be removed and replaced periodically, but the cleaning provided by the reverse flow allows for such cleaning intervals to be prolonged.

The strainer may be made of any suitable material. In some embodiments, the strainer is made of a metal material (e.g., stainless steel, copper, aluminum, etc.). In other embodiments, the strainer may be made of a polymer material. The strainer may have a mesh size suitable for catching debris that may otherwise be recirculated into the system and clog and/or contaminate the components of the basin assembly and/or the medical device.

With continued reference to FIG. 2A, the manifold 300 may be connected to the chemical dosing system 400 for purposes of receiving the detergent, high-level disinfectant solution, and/or liquid chemical sterilant; and may be connected to a water input for purposes of inputting water from a water source. The detergent, high-level disinfectant solution, and/or liquid chemical sterilant may be mixed in the manifold 300 with the water. The manifold 400 may be used in connection with circulating the detergent, high-level disinfectant solution, and/or liquid chemical sterilant during a cleaning process, high-level disinfecting process, and/or sterilizing process.

FIG. 2B shows another exemplary embodiment of the medical device processor 100. The embodiment shown in FIG. 2B is similar to the embodiment shown in FIG. 2A, but the supply line 120 of the process indicator isolation chamber 300 connects process indicator isolation chamber 300 to the manifold 400 via the supply line 133 to the high-level disinfectant solution source and/or liquid chemical sterilant source 192. In such embodiments, the supply lines 120 and 133 may be collectively referred to as a supply line. Reference is made to the description set forth above with respect to FIG. 2A, as said features apply to FIG. 2B and will not be described in detail for the sake of brevity.

It will be appreciated that in other embodiments, the medical device processor 100 may have any suitable arrangement of valves and pumps to effectuate the flow of liquid among the basin assembly 200 and manifold 300 during a given cycle. As such, any of the pumps and any of the valves may be omitted from the system. For example, in one embodiment, valves 162, 164, 166, and 168, and pumps 140, 142, 152, and 154 may be omitted from the medical device processor.

With continued reference to FIGS. 2A and 2B, the overall operation of the medical device processor 100, including the one or more pumps and valves may be controlled by control system 500. This control may effectuate, for example, any suitable cleaning, high-level disinfecting, sterilizing, and/or rinsing process.

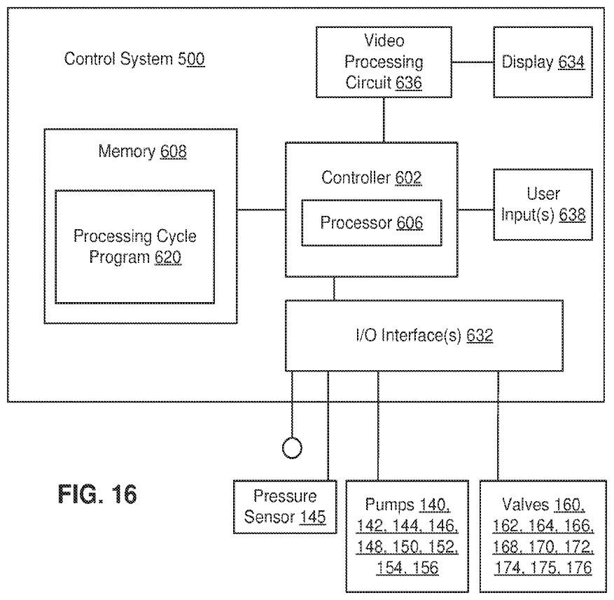

FIG. 16 shows an exemplary control system 500. Operation of the medical device processor, including the overall cycle operation, including lumen manifold pressure adjustment and strainer flush/cleaning conducted during one or more of the cycles, is controlled by the control system 500.

The controller 602 is configured to carry out overall control of the functions and operations of the control system 500. The controller 602 may include a processor 606, such as a central processing unit (CPU), microcontroller, or microprocessor. The processor 606 executes code stored in a memory (not shown) within the controller 602 and/or in a separate memory, such as the memory 608, in order to carry out operation of the various cleaning, disinfection/sterilization, and rinse cycles associated with a processing cycle; as well as lumen manifold pressure adjustment and strainer flush/cleaning. As described above, the processor may also carry out overall operation of the medical device processor 100.

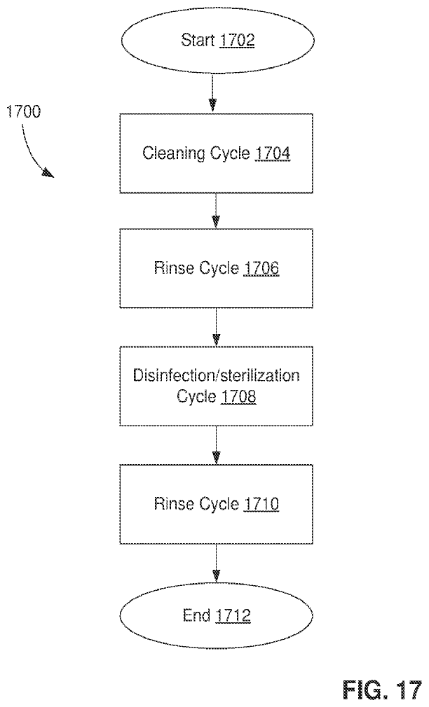

FIG. 16 shows an example in which a processing cycle program 620 is stored in the memory 608. This program may be embodied in the form of executable logic routines (e.g., lines of code, software programs, etc.) that are stored on a non-transitory computer readable medium (e.g., the memory 608) and executed by the controller 602 (e.g., using the processor 606). The processing cycle program 620 may be executed by the controller to control operation of the process cycle and implement a cleaning process and a high-level disinfecting process and/or sterilizing process (e.g., conducting a process cycle as described below with respect to FIG. 17). For example, the processing cycle program 620 may be executed by the controller to execute a cleaning cycle in which the chemical dosing system delivers a dose of liquid for the cycle. As another example, the processing cycle program 620 may be executed by the controller to execute a high-level disinfection cycle in which the chemical dosing system delivers a dose of liquid for the cycle. As another example, the processing cycle program 620 may be executed by the controller to execute a sterilization cycle in which the chemical dosing system delivers a dose of liquid for the cycle. As another example, the processing cycle program 620 may be executed by the controller to adjust and maintain lumen manifold pressure during a cleaning cycle, a disinfection/sterilization cycle, and/or a rinse cycle. As another example, the processing cycle program 620 may be executed by the controller to control pumps and valves to control flow of water in the manifold 300 to clean the strainer at the beginning of a cleaning cycle, a disinfection/sterilization cycle, and/or a rinse cycle.

The memory 608 may be, for example, one or more of a buffer, a flash memory, a hard drive, a removable media, a volatile memory, a non-volatile memory, a random access memory (RAM), or other suitable device. In a typical arrangement, the memory 608 may include a non-volatile memory for long term data storage and a volatile memory that functions as system memory for the controller 602. The memory 608 may exchange data with the controller 602 over a data bus. Accompanying control lines and an address bus between the memory 408 and the controller 602 also may be present. The memory 608 is considered a non-transitory computer readable medium.

The control system 500 may further include one or more input/output (I/O) interface(s) 632. The I/O interface(s) 632 may be in the form of one or more electrical connectors and may connect the controller 602 to one or more sensors, pumps, motors, valves, or other components. For example, as shown, the I/O interface(s) 632 is connected to the pressure sensor 145 and the controller 602 may receive and process a signal received from the sensor via the I/O interface. As also shown, the I/O interface(s) 632 connect the controller to one or more of the pumps 140, 142, 144, 146, 148, 150, 152, 154, 156 and one or more of the valves 160, 162, 164, 166, 168, 170, 172, 173, 174, 175, 176 of the system 100, and the controller 602 may control operation of one or more of the pumps and valves.

The control system 500 may include a display 634. In some embodiments, the display 634 can display information such as the state/connection status of the process indicator isolation chamber 300. The display 634 may be a lighted display (e.g., a backlit liquid-crystal display (LCD) or organic light-emitting diode (OLED) display). The display 634 may be coupled to the controller 602 by a video processing circuit 636 that converts image and/or video data to an image and/or video signal used to drive the display 634. The video processing circuit 636 may include any appropriate buffers, decoders, video data processors and so forth. The control system 500 may include one or more user inputs 638 for receiving user input for controlling operation of the control system 500. Exemplary user inputs 638 include, but are not limited to, a touch input that overlays the display 634 for touch screen functionality, one or more buttons such as those included on the handle or in a different location, and so forth. The one or more user inputs 638 may, for example, allow a user to confirm that user action has been taken in response a warning or prompt issued and displayed on the display.

FIG. 17 is a flowchart showing an exemplary processing cycle. This process may be executed, for example, in a processing cycle in which both a cleaning cycle and a disinfection and/or sterilization cycle are conducted. The control system 500 may control the medical device processor to effectuate the processing cycle. It will be appreciated that while control is described with respect to the exemplary medical device processor 100 shown in the figures, in other embodiments the medical device processor 100 may have any suitable arrangement of valves and pumps to effectuate the flow of liquid among the basin assembly 200 and manifold 300 during a given cycle, and the control system 500 may control the valves and pumps in an appropriate manner to effectuate the cycle.

In advance of the process, a user may place one or medical devices to be processed in the basin. The medical device can be hooked up to one or more connections and arranged in a predetermined manner for processing. Connection may be made using custom flow adapters, which allow fluid and/or air to flow into the endoscope from the lumen manifold. The user may also prepare the process indicator isolation chamber by inserting one or more process indicators into the chamber.

At step 1702, a process cycle is stated. This may be done, for example, by receiving a command from a user via the user inputs 638.

At step 1704, a cleaning cycle is conducted. During the cleaning cycle, the control system 500 controls pump 154 and/or valve 174 to introduce a predetermined amount of water, and controls pump 150 and/or valve 170 of the chemical dosing system 400 to introduce a predetermined amount of detergent. The cleaning cycle is conducted in a manner that the detergent containing solution is cycled through the basin and manifold for a predetermined amount of time by introducing the solution from the manifold to the basin via the spray bar(s) and nozzle(s), by injecting the solution into the channel(s) of the medical device via the connector(s), and by draining the solution from the basin back to the manifold. At the end of the cleaning cycle, the spray bar(s), nozzle(s), and connector(s) are deactivated and the solution is drained from the basin and removed from the system via drain line.

At step 1706, a rinse cycle is conducted. During the rinse cycle, the controller controls pump 154 and/or valve 174 to introduce a predetermined amount of water. The rinse cycle is conducted in a manner that the water is cycled through the basin and manifold for a predetermined amount of time by introducing the water from the manifold to the basin via the spray bar(s) and nozzle(s), by injecting the water into the channel(s) of the medical device via the connector(s), and by draining the water from the basin back to the manifold. At the end of the rinse cycle, the spray bar(s), nozzle(s), and connector(s) are deactivated and the water is drained from the basin and removed from the system via drain line.

At step 1708, the disinfection/sterilization cycle is conducted. During the disinfection/sterilization cycle, the control system 500 controls pump 154 and/or valve 174 to introduce a predetermined amount of water and controls pump 152 and/or valve(s) 172, 173 of the chemical dosing system 400 to introduce a predetermined amount of disinfectant/sterilant. The disinfection/sterilization cycle is conducted in a manner that the detergent containing solution is cycled through the basin and manifold for a predetermined amount of time by introducing the solution from the manifold to the basin via the spray bar(s) and nozzle(s), by injecting the solution into the channel(s) of the medical device via the connector(s), and by draining the solution from the basin back to the manifold. During this process, the valve 160 and/or pump 140 may also be controlled such that the solution is introduced into the chamber 298. The chemical indicator test strips, spore test strips, and/or biological indicators included therein are brought into contact with the solution to verify disinfection or sterilization efficacy. At the end of the disinfection/sterilization cycle, the spray bar(s), nozzle(s), and connector(s) are deactivated and the solution is drained from the basin and removed from the system via drain line.

At step 1710, a rinse cycle is conducted. During the rinse cycle, the controller controls pump 154 and/or valve 174 to introduce a predetermined amount of water. The rinse cycle is conducted in a manner that the water is cycled through the basin and manifold for a predetermined amount of time by introducing the water from the manifold to the basin via the spray bar(s) and nozzle(s), by injecting the water into the channel(s) of the medical device via the connector(s), and by draining the water from the basin back to the manifold. At the end of the rinse cycle, the spray bar(s), nozzle(s), and connector(s) are deactivated and the water is drained from the basin and removed from the system via drain line.

At step 1712, the process ends. Following the process, a user can remove the processed medical device(s) and can also remove the one or more process indicators to verify disinfection or sterilization efficacy.

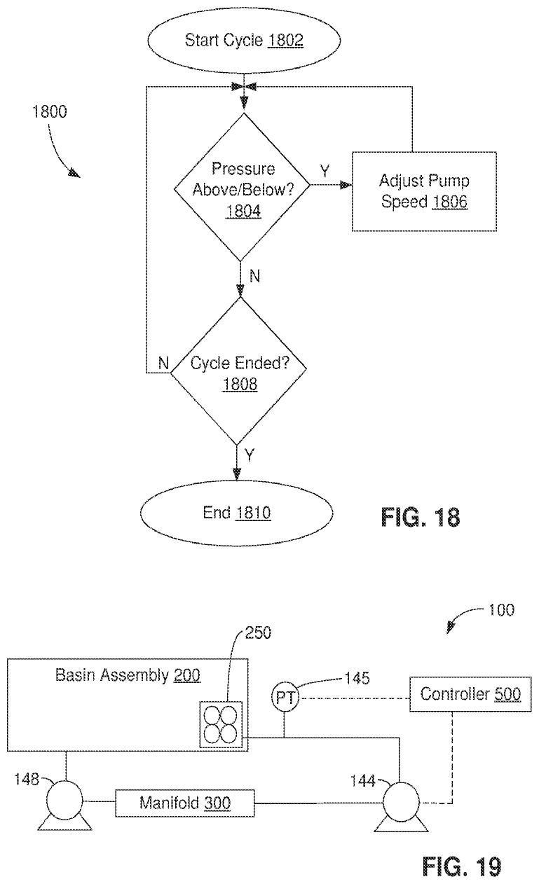

FIG. 18 is a flowchart showing an exemplary process of adjusting pressure at the lumen manifold 250. This process may be executed, for example, during any and/or each of a cleaning cycle, a disinfection/sterilization cycle, and/or a rinse cycle (for example, any of the cycles described in the process flowchart shown in FIG. 17). As described above in connection with the exemplary processing cycle, in advance of the process, a user may place one or medical devices to be processed in the basin. The medical device can be hooked up to one or more connections and arranged in a predetermined manner for processing. Connection may be made using custom flow adapters, which allow fluid and/or air to flow into the endoscope from the lumen manifold. FIG. 19 is also a simplified schematic showing parts of the medical device processor for illustration in connection with the process described in FIG. 18.

At step 1802, a cycle (e.g., rinse cycle, cleaning cycle, disinfection cycle, or sterilization cycle) is started. During the cycle, fluid flows into the variable speed pump through various tubes under gravity or may be pumped by another pump. The variable speed pump is controlled by the controller to pressurize the fluid and push it into the endoscope through the manifold. The variable speed pump is operated at a first predetermined speed and the pressure of the fluid is also detected by a pressure sensor downstream of the variable speed pump. In some embodiments, the pressure sensor is connected directly to the lumen manifold. The pressure sensor is also connected to the controller and receives pressure information.

At step 1804 it is determined by the controller whether the pressure is above/below a predetermined value or outside of a predetermined range. If yes, the process proceeds to step 1806 where the controller controls the variable speed pump by adjusting the pump speed. If the pressure is above the predetermined value or range, the controller decreases the speed of the variable speed pump. If the pressure is below the predetermined value or range, the controller increases the speed of the variable speed pump. The process then proceeds back to step 1804. If no, the process proceeds to step 1808.

At step 1808, it is determined whether the cycle has ended. If yes, the process proceeds to step 1810 where the process ends. If no, the process proceeds back to step 1804.

The process allows for pressure to be regulated and maintained at the lumen manifold and in the endoscope, independent of endoscope type (diameter of endoscope lumens/channels and number of endoscope lumens/channels), and also will work for endoscopes that may be released in the future. The process may reduce the overall number of custom manifolds and/or inline flow reducers/restrictors, and may eliminate the need for requalification or recertification of the AER for purposes of processing new devices.

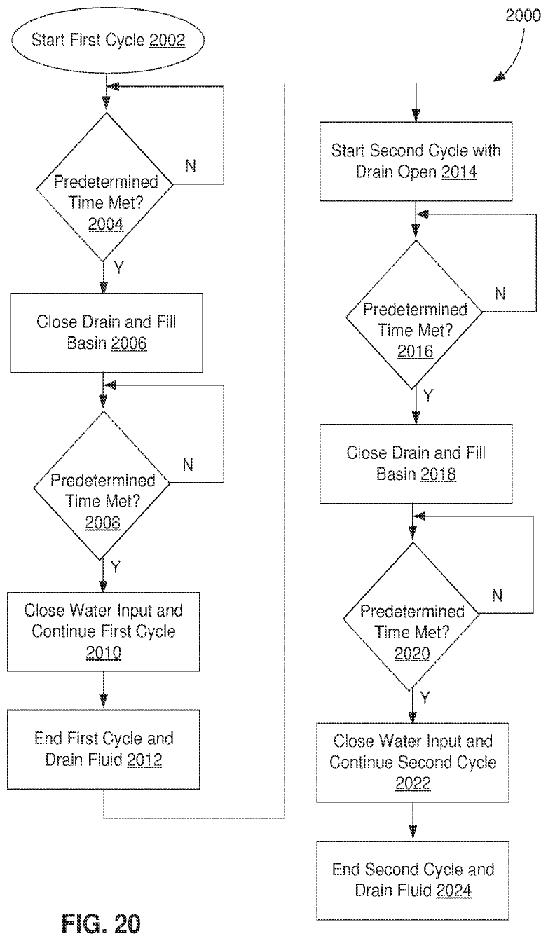

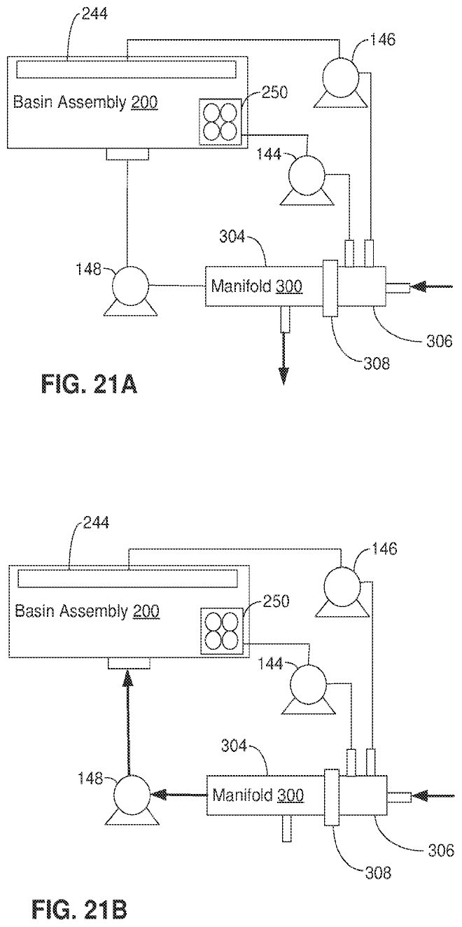

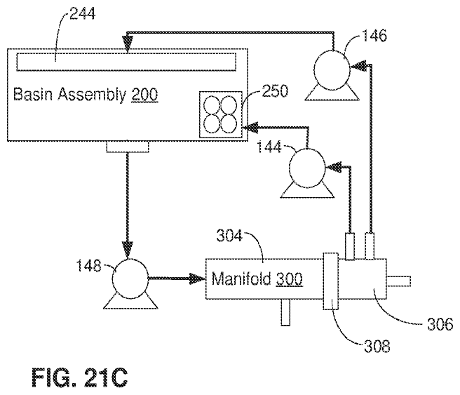

FIG. 20 is a flowchart showing an exemplary straining process. This process may be executed, for example, during any and/or each of a cleaning cycle, a disinfection/sterilization cycle, and a rinse cycle (for example, any of the cycles described in the process flowchart shown in FIG. 17). FIGS. 21A-21C are also simplified schematics showing parts of the medical device processor for illustration in connection with the process described in FIG. 20. FIG. 21A schematically exemplifies flow during the beginning of a cycle in which both the drain and water input are both open. FIG. 21B schematically exemplifies flow during the filling of the basin. In FIG. 21B, the drain is closed and the water input is open. FIG. 21C schematically exemplifies flow during a cycle, subsequent to the basin being filled. In FIG. 21C, the drain and water input are both closed. While the flowchart in FIG. 20 describes the process over the course of two cycles, it will be appreciated that the straining process may be conducted for a single cycle (e.g., steps 2002-2012). Also, in embodiments where the overall processing performed by the medical device processor includes more than two cycles, the straining process may be conducted during more than two cycles (e.g., during each cycle).

At step 2002, a first cycle (e.g., rinse cycle, cleaning cycle, disinfection cycle, or sterilization cycle) is started. Water is introduced to fill the basin for the first cycle. During this step, the drain port is kept open for a predetermined amount of time. While the drain port is open, the water will flow in an opposite direction as compared to the normal processing flow and debris previously caught by the strainer will be removed from the strainer and flushed out down the drain. Reference is made to the flow shown in FIG. 21A.

At step 2004, it is determined whether the predetermined amount of time has been met. If no, the controller continues to track the time against the predetermined amount of time. If yes, the process proceeds to step 2006 and the drain is closed after a predetermined amount of time and the basin is filled. Reference is made to the flow shown in FIG. 21B, which shows flow after the drain is closed.

At step 2008, it is determined whether the predetermined amount of time has been met. If no, the controller continues to track the time against the predetermined amount of time. If yes, the process proceeds to step 2010 where the water input is closed and the cycle continues. During the cycle, fluid is cycled through the manifold and the inline strainer filters the fluid. Reference is made to FIG. 21C, which shows flow after the drain and the water input are closed.

At step 2012, the first cycle ends. The fluid is drained from the basin and through the drain port of the manifold. Flow of the fluid into the manifold during draining is that which is shown in FIG. 21C.

At step 2014, a second cycle is started. Water is introduced to fill the basin for the first cycle. During this step, the drain port is kept open for a predetermined amount of time. While the drain port is open, the water will flow in an opposite direction as compared to the normal processing flow and debris caught by the strainer from the first cycle will be removed from the strainer and flushed out down the drain. Reference again is made to the flow shown in FIG. 21A.

At step 2016, it is determined whether the predetermined amount of time has been met. If no, the controller continues to track the time against the predetermined amount of time. If yes, the process proceeds to step 2018 and the drain is closed after a predetermined amount of time and the basin is filled. Reference again is made to the flow shown in FIG. 21B, which shows flow after the drain is closed.

At step 2020, it is determined whether the predetermined amount of time has been met. If no, the controller continues to track the time against the predetermined amount of time. If yes, the process proceeds to step 2022 where the water input is closed and the cycle continues. During the cycle, fluid is cycled though the manifold and the inline strainer filters the fluid. Reference is made to FIG. 21C, which shows flow after the drain and the water input are closed.

At step 2024, the second cycle ends. The fluid is drained from the basin and through the drain port of the manifold. Flow of the fluid into the manifold during draining is that which is shown in FIG. 21C.

In accordance with the process, every time the unit fills and drains, the strainer in the manifold can be cleaned of debris.

While the exemplary process is disclosed as conducting the filling step based on a predetermined amount of time, it will be appreciated that in other embodiments, the filling may be controlled by other means such as level sensor (not shown) located in the basin and/or a flow meter that is used by the control system for determine when to close the water input.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A basin assembly for a medical device processor, comprising:
a basin tub comprising a bottom surface, one or more side surfaces, and a raised island extending from the bottom surface, the bottom surface, the one or more side surfaces, and the raised island defining an interior volume of the basin tub, wherein:
the one or more side surfaces define a first side, a second side opposed the first side, a third side connecting the first and second sides, and fourth side connecting the first and second sides and opposed the third side; and
the basin tub comprises:
one or more lumen connector ports proximate the third side;
a control handle area proximate an intersection of the first and third sides, the control handle area shaped so that a control handle disposed in the control handle area is oriented with its ports proximate the lumen connector ports;
a light guide connector area located proximate an intersection second and third sides, the light guide connector area shaped so that a light guide connector disposed in the light guide connector area is oriented with its light guide prong distal the internal lumen connector ports;
a plug area is located on a top surface of the raised island; and
a tube/cord area located proximate the second and fourth sides and surrounding at least a portion of the raised island.

2. The basin assembly of claim 1, wherein the basin assembly comprises a drain extending through the bottom surface of the basin tub located in the control handle area.

3. The basin assembly of claim 1, further comprising raised portions at the bottom surface of the basin tub in at least one of the control handle area, light guide connector area, or tube/cord areas.

4. The basin assembly of claim 1, further comprising raised portions at the one or more side surfaces of the basin tub.

5. The basin assembly claim 3, wherein a height of the raised features is within a range of 3 mm to 15 mm, a width of the raised features is within a range of 5 mm to 25 mm, and a radius of the raised features is within a range of 3 mm to 15 mm.

6. The basin assembly of claim 1, wherein a top surface of the raised island is a contoured surface configured to retain a plug.

7. The basin assembly of claim 1, further comprising:
a control handle icon located at the bottom surface in the control handle area indicating a control handle and an orientation at which a control handle is to be disposed in the control handle area;
a light guide connector icon located at the bottom surface in the light guide connector area indicating a light guide connector and an orientation at which a light guide connector is to be disposed in the control handle area; and
a plug icon located at the raised island surface in the plug area indicating a plug and an orientation at which a plug is to be disposed in the plug area.

8. The basin assembly of claim 7, wherein at least one of the control handle icon, light guide connector icon, or plug icon are raised icons formed as a part of the basin tub surface.

9. The basin assembly of claim 7, wherein at least one of the control handle icon, light guide connector icon, or plug icon are recessed icons formed as a part of the basin tub surface.

10. The basin assembly of claim 7, wherein at least one of the control handle icon, light guide connector icon, or plug icon are printed, painted, or adhered to the basin tub surface.

11. The basin assembly of claim 1, further comprising a vent port extending through the side wall of the basin tub, the vent port comprising:
a vent wall comprising first and second opposed surfaces and holes extending therethrough;
an elastomeric umbrella-type diaphragm valve mounted to the vent wall and in contact with the first opposed surface distal the interior volume of the basin tub;
a shielding extending from the second opposed surface, the shielding comprising a shielding wall and a side wall extending from the shielding wall and connecting the shielding wall to the vent wall, wherein the shielding wall is spaced apart from the vent wall and covers the holes when the basin vent port is viewed in a direction orthogonal to the second opposed surface of the vent wall.

12. The basin assembly of claim 1, further comprising a vacuum break port extending through the side wall of the basin tub, the vacuum break port comprising:
a vent wall comprising first and second opposed surfaces and holes extending therethrough, the first opposed surface distal the interior volume of the basin tub, the second opposed surface proximate the interior volume of the basin tub;
an elastomeric umbrella-type diaphragm valve mounted to the vent wall and in contact with the second opposed surface; and
a shielding extending from the second opposed surface, the shielding comprising a shielding wall and a side wall extending from the shielding wall and connecting the shielding wall to the vent wall, wherein the shielding wall is spaced apart from the vent wall and covers at least a portion of the diaphragm as viewed in a direction orthogonal to the second opposed surface of the vent wall, wherein the shielding wall comprises a cutout that exposes a portion of the diaphragm when viewed in the direction orthogonal to the second opposed surface of the vent wall.

13. A method of processing a medical device, comprising:
performing a cycle in a medical device processor, the medical device processor comprising:
a basin assembly comprising a basin tub and one or more connectors configured to connect to a medical device processed in the medical device processor;
a supply line connected to the one or more connectors for supplying fluid to the medical device;
a variable speed pump connected to the supply for pumping the fluid;
a pressure sensor downstream from the variable speed pump and configured to detect a pressure of the fluid pumped from the variable speed pump to the one or more connectors; and
a controller coupled to the variable speed pump and the pressure sensor and configured to control the speed of the variable speed pump;
controlling the variable speed pump to operate at a first predetermined speed;
determining that a pressure detected by the pressure sensor is above or below a predetermined value or outside of a predetermined range during the cycle; and
controlling the variable speed pump to operate at a second predetermined speed.

14. The method of claim 13, wherein the medical device processor comprises a lumen manifold comprising the one or more connectors.

15. A method of processing a medical device, comprising:
starting a cycle in a medical device processor, the medical device processor comprising:
a basin assembly comprising a basin tub, one or more fluid inputs, and a basin drain;
a manifold comprising a front manifold portion and a back manifold portion coupled to the front manifold portion, the front manifold portion and the back manifold portion forming a flow path therethrough, and a strainer disposed between the front manifold portion and the back manifold portion in the flowpath;
a return line connecting the basin drain to the front manifold portion; and
a supply line connecting the back manifold portion to the basin assembly fluid inputs;
wherein the front manifold portion comprises a drain port and the rear manifold portion comprises a water input port;
introducing water into the water input port;
maintaining the drain port in an open position for a predetermined amount of time so that water input from the water input port flows through the strainer in a first direction toward the drain port and exits the manifold;
closing the drain port after the predetermined amount of time;
closing the water input port after a predetermined amount of water is introduced; and
conducting the cycle wherein the flow of water through the manifold is in a second direction opposite the first direction.

16. The method of claim 15, further comprising draining the water via the drain port upon completion of the cycle.

17. The method of claim 15, wherein the one or more fluid inputs of the basin comprises one or more nozzles, and one or more connectors configured to connect to a medical device processed in the medical device processor.

18. The method of claim 15, wherein the front manifold portion comprises one or more inlet ports for inputting one or more of detergent solution, disinfectant, or sterilization solution.

19. The method of claim 15, wherein the back manifold portion comprises one or more outlet ports connectable to the one or more fluid inputs of the basin assembly.

20. The method of claim 15, wherein the strainer is a metal mesh strainer.

21. (canceled)
